(19) United States
(12) Patent Application Publication
(10) Pub. No.: US 2011/0160751 A1
(43) Pub. Date: Jun. 30, 2011

(54) EXTRALUMINAL STENT TYPE PROSTHESIS FOR ANASTOMOSIS

(76) Inventor: Luiz Gonzaga Granja Filho, Recife/Pe (BR)

(21) Appl. No.: 12/303,650

(22) PCT Filed: Jun. 6, 2007
(86) PCT No.: PCT/BR2007/000148

§ 371 Date: Mar. 16, 2011

(30) Foreign Application Priority Data
Jun. 6, 2006 (BR) PI 0603437-3

(51) Int. Cl. A61B 17/11

(52) U.S. Cl. 606/153

(57) ABSTRACT

An external stent type prosthesis is provided that is extraluminal, with at least one tubular member, interconnectable in an upper end, and inflatable by a balloon with a central lumen, single but with multiple projections in equal plurality of tubular members of the stent which is adjusted in its interior, for side-to-side, end-to-end, end-to-side anastomosis without clamping and sutureless, or with expeditious clamping and sutureless, where the vascular graft, or anastomotic trunk, or any other grafts, inserted in the lumen of the balloon and prosthesis, comprising a distensible mesh and after being coated with graft, this mesh is expansible by the balloon until the necessary gauge to keep the graft wall joined together and sealed in relation to the organ wall, that can contain a bag suture around the place where the anastomosis is made.

### FIELD OF INVENTION

The present invention relates to an extraluminal, external ‘stent’ type prosthetic device, to perform a sutureless and without clamping anastomosis, or with expeditious clamping (reserved to normal walls organs) and sutureless, where the vascular graft, or any other, is inserted within the prosthesis lumen. After being coated with graft, stent type prosthesis is expanded by a balloon. The stent can have a single or multiple intraluminal part, allowing wide anastomosis, simultaneously, without clamping and sutureless, with grafts joined together to an anastomotic trunk at the same time, but isolated and separated from one another, each one with its intraluminal part.

### DESCRIPTION OF THE PRIOR ART

A prior art presents several trials provide solutions for anastomotic devices projected to correct vascular abnormalities, which present the following typical features:

The North-American U.S. Pat. No. 3,254,650, of Jun. 7, 1966, describes a method and devices to execute anastomosis procedures by applying with adhesive two separated connectors in a body member and removing this body member portion contained among the connectors, joining the said connection devices for joining the remaining portions of the body member.

The U.S. Pat. No. 3,265,069, of Aug. 9, 1966, describes devices or instruments for use by surgeons in reunion of body ducts, which in the course of operations were separated. The instruments comprise a pair of elongated similar elements and articulatedly connected, in an intermediary manner, and with an support for finger retention in a distal end, comprising a generally cylindrical shape with a cylindrical channel that passes through it in the other distal end, in order to receive tubular body ducts kept by the instrument while the body ducts are reconnected.

U.S. Pat. No. 3,774,615, of Nov. 27, 1973, describes a device to connect the end of interrupted tubular organs without sewing, comprising a connecting ring on which the end of the interrupted organ are pulled, the ring is preferably locked up by a fixation resource. The ring and fixation resource are made of inert material, and preferably a hydrophile gel that can be dilated until its equilibrium or can be a hydrogel incompletely dilated, which is submitted to additional dilatation where it is applied. The connecting ring can be supplied with a groove and can be placed in a ring shaped fixation resource and kept there joining it to the fixation resource in the groove or simply kept by a screw. Two connection rings can also be used and kept joined by a coupling member.

The document U.S. Pat. No. 4,366,819, of Jan. 4, 1983, describes an anastomotic joint for surgery with a graft of coronary artery deviance comprising a mounting of four elements including a cylindrical tube with at least one locking indentation of ring flange in one influx end and a plurality of grooves of locking ring in a flow end; a ring flange with a central opening and a plurality of long and short spigots, the long spigots are engaged in the locking indentation, with a graft engaged among them; a fixation ring with a central opening and a plurality of spigots positioned around the opening; and a locking ring with a opening with a plurality of locking ring edges for engaging with the locking ring grooves. In surgical implants, an aortic wall with a hole engages between the ring flange and the fixation ring and is kept in this position by spigots of the fixation ring, and the four elements engage together forming an integral anastomotic joint. A first alternate modality includes an anastomotic joint of three elements with a combination of fixation ring and locking ring. A second alternate modality includes an anastomotic joint of four elements with a slightly jolted end in a influx end, exposing the graft material in the anastomotic “ostium”.

Other prior arts are equally mentioned, base don some information of “The Cardiothoracic Surgery Network”. The “Simmetry Aortic Connector System”, developed by St. Jude Medical, is a connector made with nitinol, selected by vein diameter with an adventitia removed to allow adjust of the connector and to prevent its displacement by the blood current. Then, the device may make an angle of 90° with the aorta. Among the disadvantages, there is the fact that it can be used only in extreme cases due to the difficult usage of this technique; it did not obtain a satisfactory result in many surgeries and it is being drowned out of market by the manufacturer; it is not applicable in calcified aorta; presents suture; presents contact with blood flow (foreign body); it does not widen the anastomosis area (restrictive anastomosis); performs only one anastomosis at a time; it is a product restrict to end-to-side anastomosis; a great mobilization of the venous graft occurs, damaging it, and can eventually form thrombus; there is a risk of perforation of the posterior wall of aorta; and the adventitia is removed (most resistant vascular layer).

Other known device is the PAS-Port™System, a device used in 3 steps, and the vein wall is mounted over the device and is manually reversed on it, by tool and adapted to aorta with a angle of 90°. The method alerts that the surgeon shall select with due care the point of aorta and the vein size. The device is made of stainless steel and is available in only one size that allows the use of veins with external diameter of 4 to 6 mm, aorta with an internal diameter of 18 mm. It is available in only one size, limiting its applicability. As disadvantages of this prior art, the device has contact with blood flow (foreign body); it does not widen the anastomosis area (restrictive anastomosis); it uses veins with external diameter of 4 to 6 mm and aorta with an internal diameter of 18 mm; it does not perform multiple nor visceral anastomosis; it performs just only end-to-side anastomosis; a great mobilization of the used biological graft occurs, damaging its inner layer, which generates the formation of thrombus; there is a big risk of kinking at the origin (angle of 90°) and risk of posterior wall perforation in the aorta at the moment the device is introduced under its light; the suture is substituted with disadvantages by stainless steel (9 pins, distant among them, maximizing the risk of bleeding).

Also as prior art, there is the CorLink Device, currently commercialized by Ethicon/Johnson & Johnson, that allows the creation of anastomosis between the ascending aorta and a saphenous vein segment. Aortic Anastomotic Device (AAD) is a self-expanded device with extra luminal nitinol constituted by a de um central cylinder with five interconnected elliptical arches and 2 groups of 5 pins in the end portion of the cylinder. The pins, after the eversion of venous walls in the device, fix the aggregate penetrating into the venous graft wall. A blade makes an opening in the wall of aorta and permits the coupling of AAD, which also fix the wall of aorta by pins. With this device: it poses a serious risk of bleeding, especially in friable aortas, thin, calcified or fibrous, restricting its applicability, also with risks, even in aortas with normal walls; in small gauge anastomosis, there is a risk of thrombosis, hyperplasia, intimal proliferation and fibrosis (reaction to foreign body type in origin of anastomosis) with consequent stenosis resulting in occlusion of anastomosis; sutures are used in some cases; there is cases of infarction caused by equipment; there is a recurring need of re-operations in patients; the device presents contact with blood flow (foreign body); it is not flexible; it does not multiple anastomosis; an inadequate mobilization of venous graft occurs, and can cause damage to its intimal layer, it could form thrombus; it is used only in extreme cases because it is a technique of complex usage; the suture is substituted by stainless steel in contact with blood flow.

Another known device is the St Jude Distal Connector that consists of a stainless steel clip mounted on a catheter, comprising a balloon for subsequent expansion and connector mounting. The catheter is introduced backward from the end, by doing a small hole in the anastomosis site, the clip fixes the vein in the hole, the catheter goes to coronary and releases the connector. The catheter is removed and a suture is done in side-to-side anastomosis. With St Jude Distal Connector, occurrence of leakage problems were detected in 20% of the used connectors; the use of a metallic clip requires due care for handling to avoid distortion in the anastomosis; late angiographies reveal smaller circular diameter of anastomosis made with o St. Jude Distal Connector, when compared to controls made with conventional suture; there is remarkably risk of bleeding and the graft is very mobilized, and lacerations can occur in its inner layer, allowing the formation of thrombus.

The HeartFlo™ is a multi-suture instrument for anastomosis with wires automatically applied in end-to-side and side-to-side anastomosis. The surgeon manually ties the suture wires (10 wires) and concludes the anastomosis similarly to the traditional process. Besides of being a product of complex handling, it makes suture in anastomosis (keeping the undesirable foreign body in the internal origin of the anastomosis) and is restricted to end-to-side and side-to-side anastomosis. There is also an excessive mobilization of graft, and can cause lesions in its intimal layer, which would be the inductor that forms the thrombus.

Another technique and known device is the Solem Graft connector, produced by the Swedish company Jomed. It is constituted by a stent made of nickel and titanium coated with polytetrafluorethylen used to connect the internal thoracic artery the left anterior descending coronary artery. The results has not been satisfactory, because it poses risk of bleeding; there is also an excessive mobilization of graft, probably damaging intimal layers, allowing the formation of thrombus; it is not flexible, by this fact, causes trauma to grafts; it does not make multiple anastomosis, at a single time; presents contact with blood flow (foreign body); and is frequent the need of-operations.

The Magnetic Vascular Positioner System is produced by Ventrica and comprises 4 magnetic rings and the anastomosis is processed by magnetic attraction of 4 ports. However, initial experimental results demonstrate leakage, also a undesired contact of materials with blood flow. On the other hand, it is necessary to be careful to avoid the capitation of excess of tissue among the magnets. With this system, there is also a need of suture in some cases; there is occurrence of infarction caused by equipment; and is frequent the need of-operations in patients; and also requires clamping.

Also, as a device known by the medical area, the Combined Anastomotic Device and Tissue Adhesive, developed by Grundeman & Borst group, combines micro mechanical technique with use of adhesive (glue). The use of this method can result in leakages and need traditional sutures; it is frequent the need of re-operation due to leakage/bleeding; and performs only one anastomosis at a time.

Finally, it is also experimentally practiced anastomosis assisted by laser, where the results are not different from conventional isolated sutures, because there is a need of suture in some cases; there is a risk of bleeding e leakage; and does not perform multiple anastomosis.

Even so divulged nowadays, anastomosis with clamper, by insecurity, and almost totality of surgeons perform conventional sutures throughout the route of anastomosis, with an intention of avoiding leakages and bleedings, it means the use of clampers just makes the procedure more expensive, once the conventional suture is also applied.

In short, the conventional anastomosis, with clamping and with suture, standardized in 1906 by Aléxis Carrel, remains the first choice for any type of anastomosis and organs to be anastomosed.

With an expectation of changing the current situation, the Brazilian patent no. PI 9706197-2, describes and claims a prosthesis for vascular anastomosis, or in any other organ or tissue, without the use of clamping and sutureless, solving, in an elegant and efficient manner, the limitations inherent to prosthesis of the above mentioned prior art, when used in vascular anastomosis performed, mainly in thin aortas, calcified and friable; or in any other application where a clamping of a vein or artery can pose excessive trauma for conditions of a given patient. The prosthesis that is subject of that request allows the embodiment of fast and safe anastomosis, without obstruction of vein or artery lumen of which anastomosis is made, also allows anastomosis in tissues, veins or arteries in bad conditions and never would accept a clamping used in conventional anastomosis. This is achieved by a generally cylindrical shaped prosthesis with a flange orthogonally extending from its external side wall, in a point in the prosthesis length between its ends; the referred flange has openings distributed around its surface. The description of the usage method and specific construction of the prosthesis is presented in the drawings of the descriptive report of that request, as well as the document C19706197-2, Certificate of Addition of the first.

Although these anastomotic devices can be presented as suited to the purposes for which they were projected, they are not so suited for the purposes of the present invention, as described herein below.

### SUMMARY OF THE PRESENT INVENTION

The present invention describes an external stent type prosthetic device, extraluminal, used for side-to-side, end-to-end and end-to-side anastomosis without clamping and sutureless, or with expeditious clamping and sutureless, where the vascular graft, or any other (intestines, urether, choledoch, trachea and bronchus, uterine tubes, urethra, deferent ducts etc; or synthetics), is introduced in the prosthesis lumen, and after coated with graft, the prosthesis is expanded by a balloon up to a determined gauge in order to maintain the graft wall joined and sealed to the organ wall in which the anastomosis performed. The graft is rotated by jacketing to cover part of the stent type prosthesis, which, when insufflated, eliminating the contact of foreign bodies within the anastomosis. The prosthesis can also present varied sizes and formats to accommodate simultaneously a varied sizes and types of grafts.

An objective of the present invention is to provide an external stent type anastomotic device, balloon expansible, allowing to regulate its gauge, exactly to the incision diameter of the organ and graft gauge.

Another objective of the invention is to provide an anastomotic device for any type of anastomosis (side-to-side, end-to-end e end-to-side) without clamping and sutureless.

Another objective of the present invention is to provide an external stent type anastomotic device, extraluminal, balloon expansible, allowing to perform any kind of anastomosis between any tubular organs without clamping.

Another objective of the present invention is to provide an anastomotic device that does not introduce any foreign body inside the anastomosis grafts.

Another objective of the present invention is to provide an external stent type anastomotic device that avoids the occurrence of graft kinking or anastomotic trunk when they emerge from the prosthesis, independently from where they are placed or at which pressure they are submitted to. It occurs due to the fact that the grafts or trunk are fixated with a circumferential distance from where the prosthesis emerges, and set free inside it.

A further objective of the present invention is to provide an external stent type anastomotic device that can be partially single or multiple intraluminal, allowing to perform multiple, wide anastomosis, simultaneously, without clamping and sutureless, with grafts joined together in an anastomotic trunk or at the same time, but isolated and separated from one another, each with its own intraluminal part.

Other features and additional objectives of the present invention will become apparent from the following descriptions. These features will be described at sufficiently detailed levels to allow the technicians of the subject matter to implement the invention. Also, it is understood that other features can be used and structural changes can be made without leaving the scope of the invention. In the accompanying drawings, like reference numbers indicate identical or like parts throughout the several views.

Therefore, the following detailed description should not be taken as limiting the scope of the present invention which is defined by the appended claims.

### BRIEF DESCRIPTION OF THE DRAWINGS

The present invention may be understood more completely by reference to the following description and appended drawings, supported by examples, in which:

FIG. 1 illustrates external stent type prosthesis before the balloon is insufflated by a syringe, still not coated with graft.

FIG. 2 illustrates external stent type prosthesis before the balloon is insufflated, already coated with graft.

FIG. 3 illustrates an external stent type prosthesis coated with graft after the balloon is expanded.

FIG. 4 illustrates the end of an end-to-side anastomosis, without clamping and sutureless, with external stent type prosthesis, after the balloon deinflation and removal.

FIG. 5A illustrates a first modality of the external stent type prosthesis mesh, with its details.

FIG. 5B illustrates a second modality of the external stent type prosthesis mesh, with its details.

FIG. 5C illustrates a third modality of the external stent type prosthesis mesh, with its details.

FIG. 5D illustrates a fourth modality of the external stent type prosthesis mesh, with its details.

FIG. 5E illustrates a fifth modality of the external stent type prosthesis mesh, with its details.

FIG. 5F illustrates a sixth modality of the external stent type prosthesis mesh, with its details.

FIG. 6 illustrates an inflatable balloon with three extensions that are insufflated simultaneously.

FIG. 7 illustrates in transversal cut, some possible configurations of the inflatable balloon extensions.

FIG. 8 illustrates a stent constituted by three tubular members, distended simultaneously by a balloon of the same form, which inflates internally.

FIG. 9 illustrates in transversal cut some possible configurations of the prosthesis with multiple tubular members.

FIG. 10 illustrates the set formed by a stent with multiple tubular members and the inflatable balloon with its multiple extensions inside them, besides the manometer and syringe for inflation.

FIG. 11 illustrates a set of stent, balloon completely coated with graft. This balloon has a perfusor for insufflation located on a half distance between the ends. This set is especially indicated to side-to-side anastomosis without clamping and sutureless, or with expeditious clamping and sutureless.

FIG. 12 illustrates the slightly expanded front anastomotic aggregate.

FIG. 13 illustrates the aspect of the anastomosis with inflatable balloon still filled.

FIG. 14 illustrates the final aspect of the anastomosis with the balloon completely collapsed, by active aspiration, enlarging the anastomosis lumen.

### DESCRIPTION OF THE PREFERRED EMBODIMENTS

With reference to the drawings, in which like reference numbers indicate identical elements throughout the several views, the figures illustrate one form of the present invention, in form of prosthesis for external stent type anastomosis, extraluminal, and the mesh types.

FIG. 1 illustrates external ‘stent’ type prosthesis, extraluminal, formed by a mesh 1 with drawing and varied sizes, insufflated by a balloon 2, with predetermined maximum sizes. The balloon 2 also with lumen, is inserted in its interior, and insufflated with syringe 3, or other method, under pressures controlled by the manometer 5.

FIG. 2 illustrates the anastomotic aggregate represented by external stent 1, balloon 2 with manometer 5, and the graft 6 that passed through the balloon lumen 2 and recovered externally, the whole extension of the stent 1, which was little and slightly dilated, assuming a form of an asymmetrical reel, with an internal flange slightly minor than the external. The graft is fixed to the stent 1, in its external end, by some simple separated points.

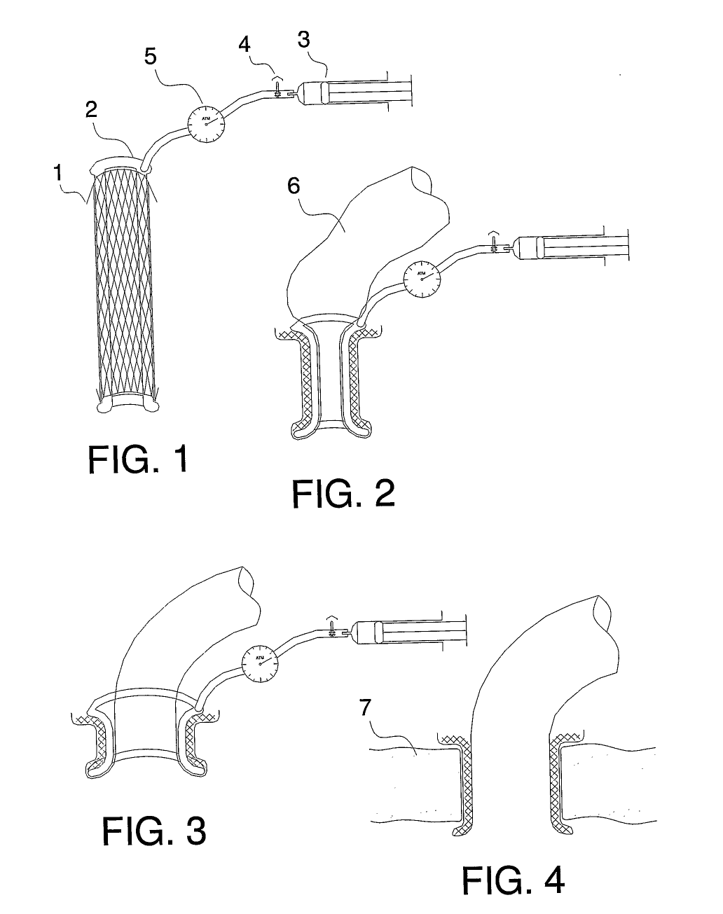

FIG. 3 and FIG. 4 illustrate the dilatation process of extraluminal the stent 1 and later removal of the balloon 2. A technical achievement to its utilization in an end-to-side anastomosis, without clamping and sutureless, in any of the two or more tubular organs could be described as follows: the balloon 2 is softly inflated and it slightly dilates the stent 1, opening the balloon 2 light. It passes through the balloon 2 light, and consequently the stent 1, the graft 6 or anastomotic trunk (diameter widening of any graft with its own segments longitudinally open, and can be in its whole extension or only in its end that covers the stent, or union, by an extraluminal suture, preferably, or any other method, of several ends of several grafts or in its whole extension). The graft 6 or trunk is everted by total and external coating of the external stent 1 (remaining on the top, because when the stent 1 is expanded, if the graft 6 is not suitably fixed, it can slip inward to a level next to the origin point of the anastomosis, it could be dangerous if the graft 6 of the stent 1 is released). Due to this fact, simple separated points shall be applied between the everted end of the graft 6 and the external end of the stent 1. A suture is prepared in a bag, simple or double opposed (preferably), with seromuscular or total points. Total points are safer in organs with calcified or friable walls 7. Besides fixating the organ layers among them, it also fixes any abnormal elements that are in its internal surface, such as atheromatous plates, thrombus, calcifications, avoiding thromboembolisms at the moment of the incision and introduction of the anastomotic aggregate. Also, the wall 7 is more resistant to laceration. It is incised in the center of the suture in a bag, a temporary digital tamponade is made, the anastomotic aggregate is introduced, and the bag suture is slightly and externally adjusted. The balloon 2 is inflated until the total expansion of the stent 1, which maximum expansibility is controlled by the diameter of the graft 6 or trunk that passed through its lumen. It is important to remark that in any moment, the expansion of the stent 1 by the balloon 2, damages or put in risk the integrity of the inner layer of the grafts 6, any of the kind. The balloon 2 compresses from inside to outward the stent 1 that will also compress from inside to outward, only the everted external surface of the graft 6 or trunk that was covered, as is easily perceived in the FIG. 3 and FIG. 4. Thus, the origin of the anastomosis stays intact, as it is mandatory. This not occurs in the existing stents, which are intraluminal and has contact with the fluid that flows. In this last case, besides the contact of the fluid with a foreign body that is the material of the stent, and also, its expansion, in 100% of the cases, it can lacerate, cut and heavily damage the inner layer of the grafts, or organs. After reaching total expansion, or maximum expansion, it is joined until the two bag sutures are totally adjusted, opposed, externally to the anastomotic aggregate, adjusting organ wall 7 to it and assuring the inexistence of anastomotic leakages. The final form assumed can be of an asymmetrical reel, as presented in the FIG. 4, or even the external flange with a straight intraluminal tubular member in the external stents with only an intraluminal extension.

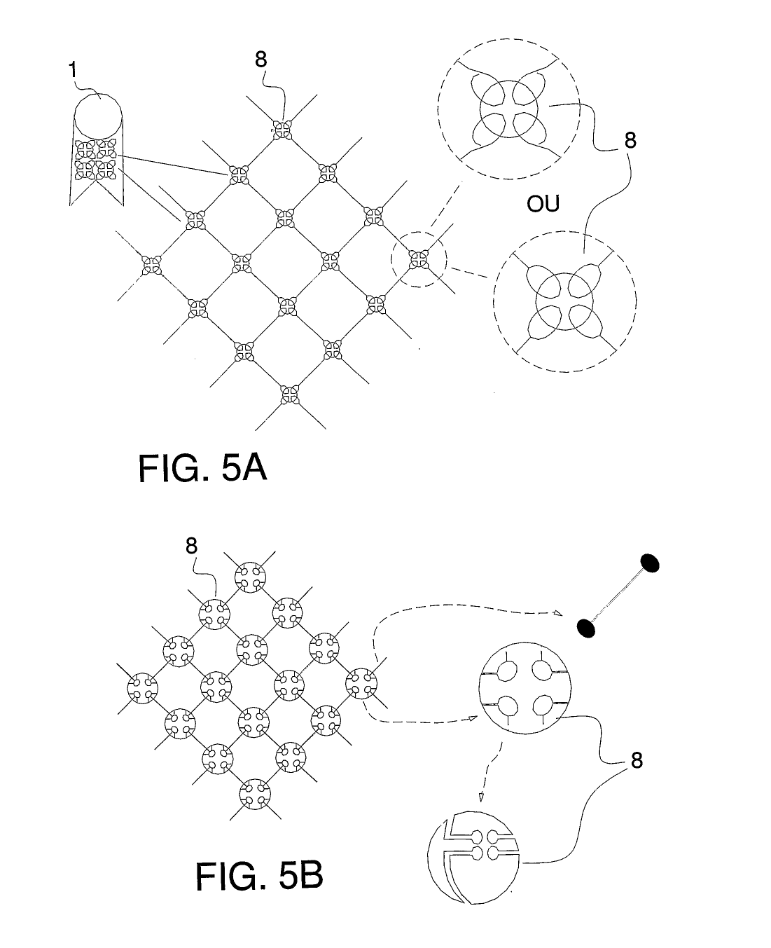

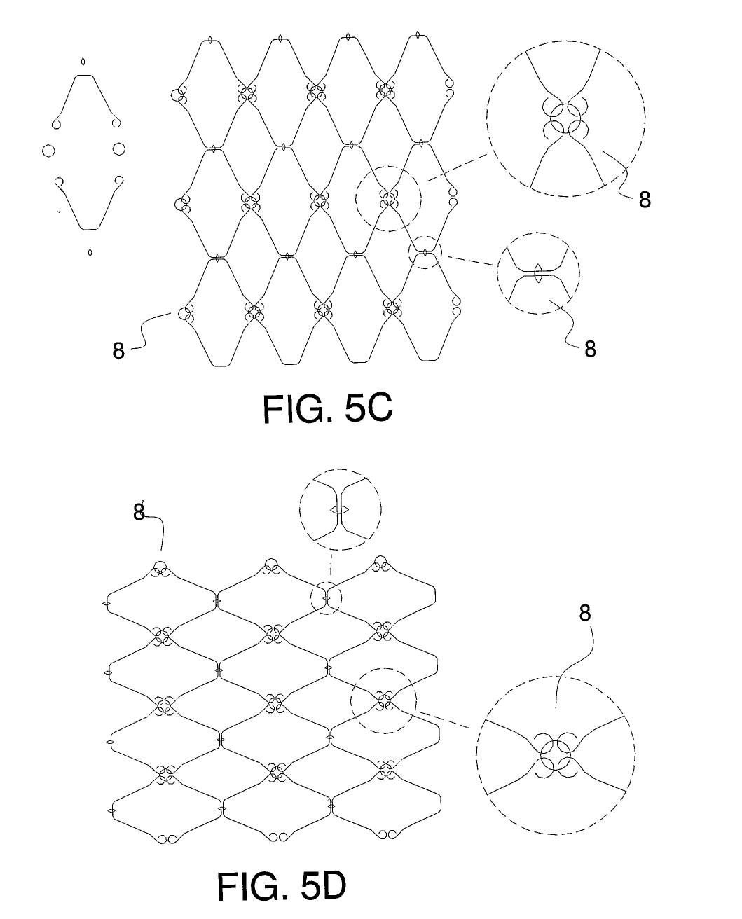

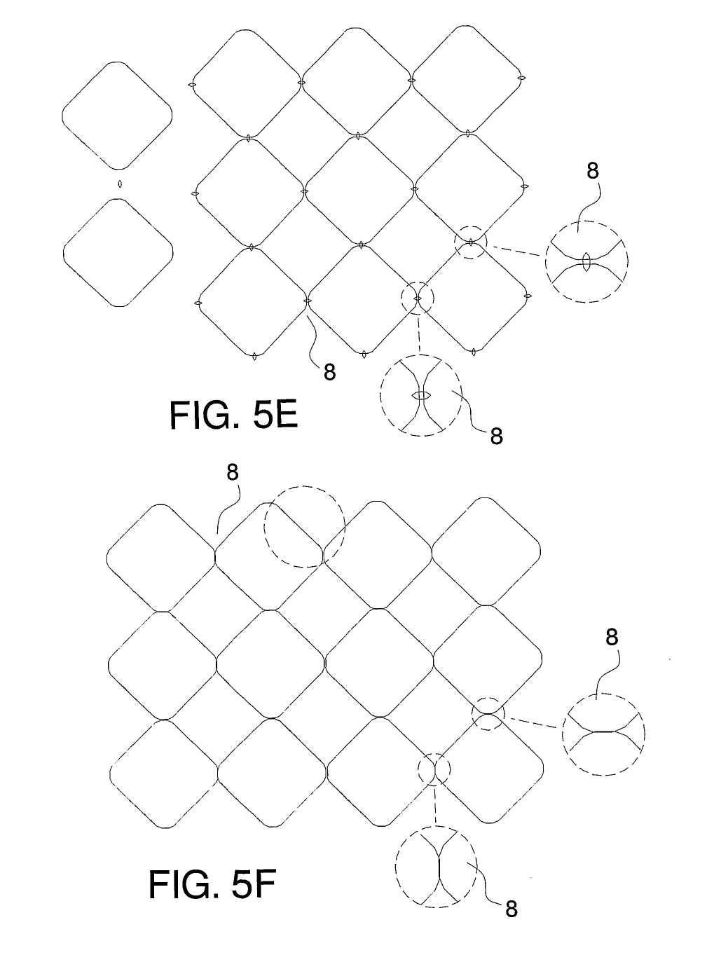

FIG. 5A and details, illustrate a form of construction of the external stent 1 mesh that can be of just the joint 8 of the twisted, open or closed ends, with four little rods through a ring. This is in longitudinal multiple form and transversally in order to form a cylinder, in idling position, the rings are transversally next one another, and are distant when it is expanded. When it is the contrary, in longitudinal direction, when idling, they are distant and when expanded, they are nearer. Therefore, the non expanded stent 1 has a minor gauge and greater extended and when expanded, it has more gauge and less extension.

FIG. 5B and details, the joint rings are substituted by grooved microspheres 8 where the four rods with shape of halters are inserted, with massive spherical ends, which enter in the microspheres grooves of the joint. After the expansion by internal insufflation by the balloon 2, the stent 2 has its diameter increased, reduces the extension and does not return spontaneously to its original form.

FIG. 5C and details, present other form of connection 8, this time by using two rings of different sizes and places, putting together also different parts of the same rod, witch are presented herein in ‘V’ form with flat vertex, once upward, once downward. The bigger ring ties-up transversally the twisted ends of four rods, and the minor ring ties-up only the flat vertex of two of these same rods, longitudinally. In the same manner, after the expansion through internal insufflation by balloon 1, the diameter increases and the extension is reduced, and it does not return spontaneously to its initial form.

FIG. 5D and details, differ from FIG. 5C only by changing the position of the ‘V’ form rods, which are laid transversally. The rings bind them equally in the same positions, the minor ring in the flat vertex, binding two rods, but transversally in this case, and the bigger ring, longitudinally binding the four twisted ends of four juxtaposed rods, two by two. In the same way, it does not return to the initial form after expanded.

FIG. 5E and details, illustrate the mesh formed by the joint 8 of the two rods, multiple, longitudinally and transversally, by little rings, which when reach the maximum expansion present a four sided polygon format. They do not deform after expansion of internal insufflation by balloon 2 and do not return to the initial position.

FIG. 5F illustrates other mesh in which the angles of the polygonal rods are joined-up 8 in a single piece, pre-molded, or by any mode, welding type, for example. In the same manner, it maintains its form after expanded.

The rods also could be circular or semicircular with any configuration of the described joints.

The stent 1 could also be prepared with a single twisted wire forming varied geometrical figures.

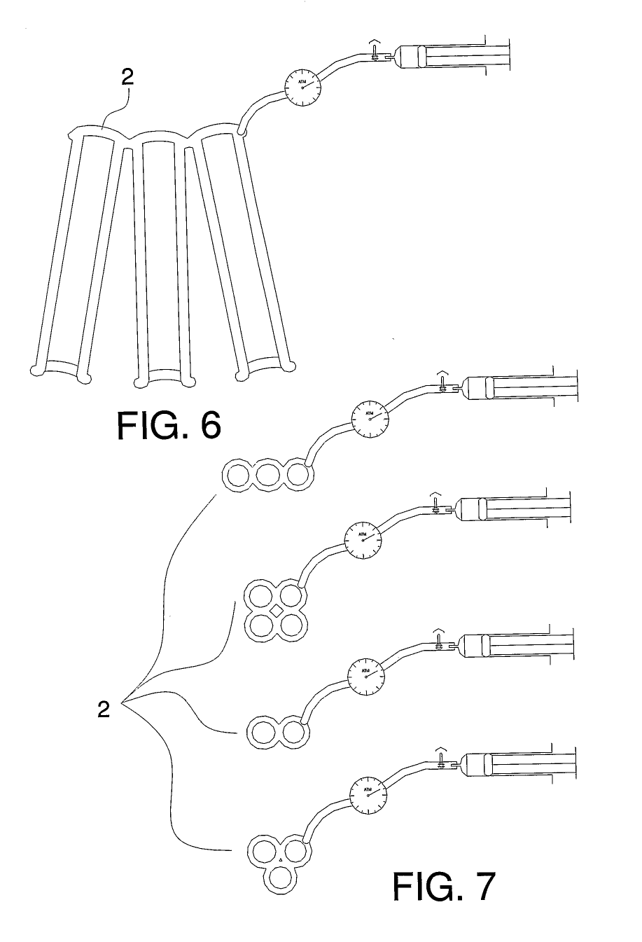

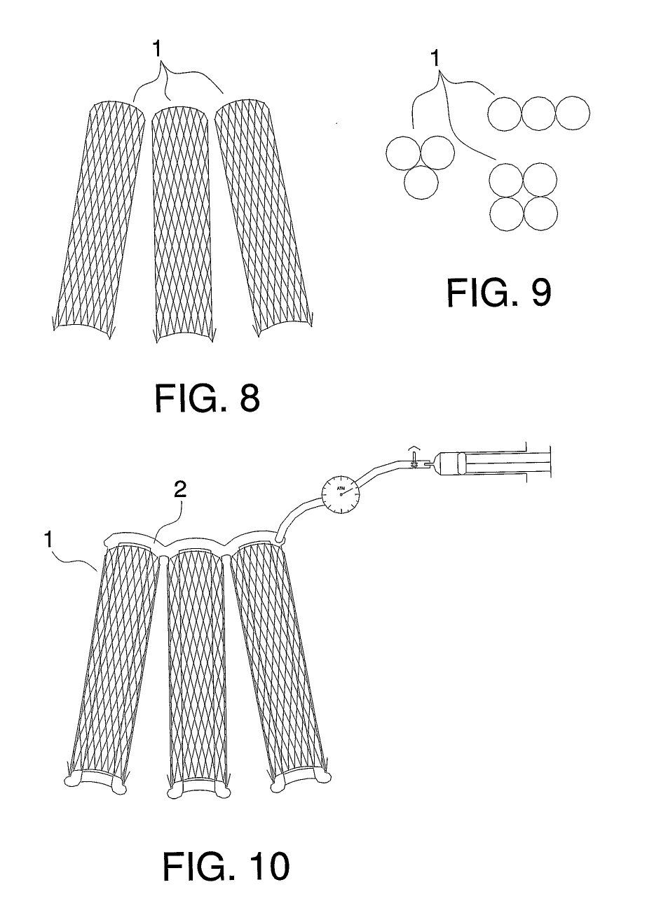

If the external stent 1 has multiple intraluminal parts, as illustrated in FIG. 8, FIG. 9 and FIG. 10, in variable forms and drawings, type, clover leaf shape, aligned, in square, rectangular, triangular etc., and arranging several grafts isolatedly, but simultaneously, also has a balloon 2 equally with multiple extensions, as illustrated in FIG. 6, FIG. 7 and FIG. 10, in equal number as the intraluminal parts, which will pass through by its lights, and also simultaneously are insufflated. The technical utilization can be exactly as described above, although other technical applications are possible. An important reason for preparing the prosthesis is that the intraluminal parts, which are separated from one another before expansion, is absolutely juxtaposed when insufflated and the everted parts of the grafts that cover them are firmly compressed one against the others. Therefore, if there is no dead spaces among them, internally and externally, there will not be blood leakages or of any flowing fluids.

One of the most important characteristics of these anastomosis, besides the absence of clamping or suture is that in any anastomosis, in which the graft 6 is everted, covering the intraluminal portion of the prosthesis and fixed by circumferential point or any method, there is no graft kinking, independently of its positioning and pressure it is submitted to. This is due to the fact that the grafts 6 were fixed distant from the local where they emerge in the prosthesis, being fully released inside the prosthesis.

An end-to-end anastomosis without clamping and sutureless can be easily prepared, with two anastomotic aggregates represented by two stents 1 and two balloons 2, with coated ends of any graft 6. In the same manner, it makes the bag suture, double, in the side walls of the organ, in the places selected for anastomosis, introducing the aggregate and expanding the stent 1 by removing the 2, and joining the bag sutures. The graft 6 is externally clamped, but the organ is not. With the same balloon 2 or other of identical of different sizing, depending on the other end to be anastomosed, after removing or cutting it (if the prosthesis and balloons are previously mounted to the grafts, to remove them, it is necessary to cut them after tractioning them outwardly from the stent 1 and graft 6), mounting the second aggregate on the other end and performing the same procedure. The organ segment that was interposed in two anastomosis can be excluded by resection or simple circumferential ligaments, deviating all the fluid through the anastomosis that has been made.

Also a side-to-side anastomosis without clamping and sutureless can be prepared even by an endovascular or videoendoscopic procedure. With a graft of two external stent 1 type prosthesis in each end, the organs to undergo a side-to-side anastomosed can even be distant. It is identical to the procedure for end-to-side anastomosis (and, in fact, there are two end-to-side anastomosis among the anastomotic aggregates and each organ).

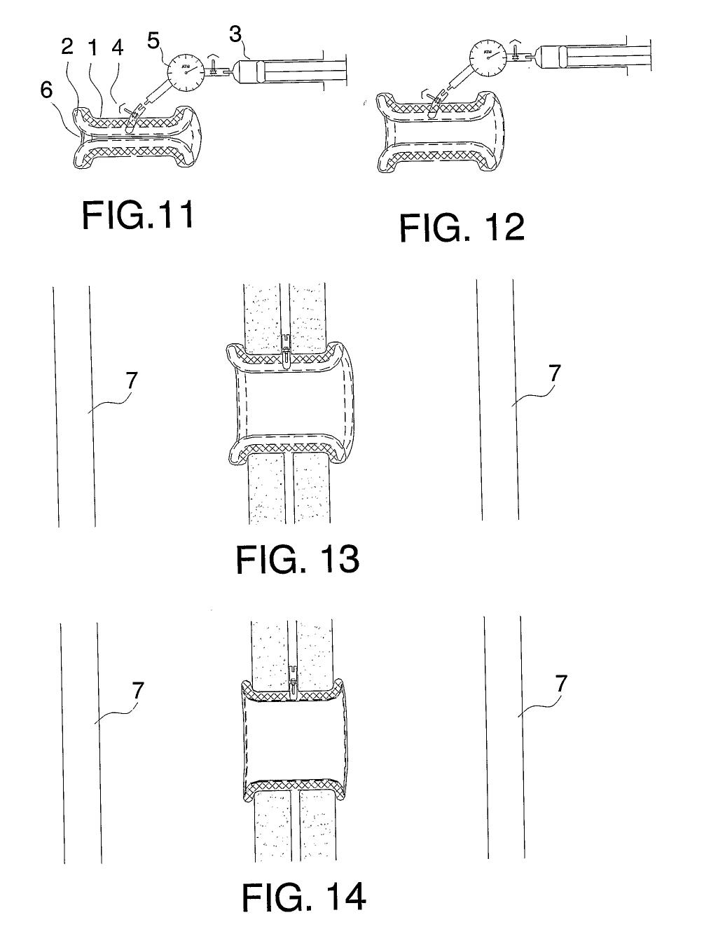

FIG. 11, FIG. 12, FIG. 13 and FIG. 14, illustrate an anastomosis with only one prosthesis, to tie together the adjacent organs in a side-to-side form. It will be completely coated with graft of any nature, which ends are in the middle, of each side of the perfusor 4 of balloon 2, in this is case has a different shape and also is completely coated with graft, except its perfusor 4, as shown in the FIG. 11. The balloon 2 has a virtual groove before its insufflation. The graft 6 passes by its light keeping it more closed still in order to avoid the bleeding or leakage of any fluid, as shown in the FIG. 11. One of the ends of the prosthesis, balloon 2 e graft 1, are introduced on the side of the organs, and the bag suture is slightly pressed, as described. The bag suture wire can transfix the graft that coated the stent 1, to avoid its displacement before the insufflation. As mentioned, there is no bleeding or leakage of any fluid. The other graft 6 is incised also in the center of the double ball suture, the other end of the anastomotic aggregate is introduced and the bag suture is equally and slightly adjusted. With the help of an assistant holding the bag sutures, the surgeon inflates the balloon 2 that distends the external stent 1, until the desired limit, permeabilizing the graft 6 by which the fluid will pass through. The bag sutures are definitely joined, as illustrated in FIG. 13. At last, the balloon 2 is deinflated and aspirated until its total collapse, in order to occupy the minor possible area and, keeping it where it, as represented in the FIG. 14. Here lies then the importance of the balloon 2 constituted of thinner and resistant walls as possible, and biocompatible material, in order to resist to high pressure of insufflation as soon it is aspirated, to not represent a remarkably loss of internal diameter of the anastomosis. Thus, what keeps the anastomosis open until it reaches the previously determined maximum diameter in the stent 1, which once expanded, changes its shape, diminishes the extension and increases its diameter and does not collapses spontaneously.

1. Prosthesis for extraluminal, external stent type (1) anastomosis comprising: at least one tubular body wherein the at least one tubular body (1) comprises a distensible and elastic mesh; and also comprises an inflatable balloon with lumen (2), having at least one insufflable (2) extension, internally arranged in relation to the mesh that forms the tubular member (1).

2. Prosthesis, according to a claim 1, wherein the at least one insufflable extension (2) can inflate and distend individualized stents (1).

3. Prosthesis, according to a claim 1, wherein the at least one insufflable extension (2) is removable.

4. Prosthesis, according to a claim 1, wherein the at least one insufflable extension (2) is not removable.

5. Prosthesis, according to claim 1, wherein the at least one insufflable extension (2) is insufflated by a syringe (3) and comprises a pressure gauge (5).

6. Prosthesis, according to claim 1, wherein the at least one insufflable extension (2) can be insufflated with a non-viscous fluid.

7. Prosthesis, according to claim 1, wherein graft kinking (6) does not occur, regardless of where it is placed and at which pressure it is subjected to.

8. Prosthesis, according to claim 1, wherein the tubular mesh represents a predetermined set of final gauge.
